(19) United States
(12) Patent Application Publication
(10) Pub. No.: US 2022/0067435 A1
(43) Pub. Date: Mar. 3, 2022

(54) APPARATUS AND METHOD FOR X-RAY DATA GENERATION

(71) Applicant: ELECTRONICS AND TELECOMMUNICATIONS RESEARCH INSTITUTE, Daejeon (KR)

(72) Inventors: Jeongwon LEE, Daejeon (KR); Seung-Hoon CHAE, Daejeon (KR); Seunghwan KIM, Daejeon (KR); Yoon Seon SONG, Daejeon (KR); SOOYEUL LEE, Daejeon (KR); Ji Wook JEONG, Daejeon (KR)

(21) Appl. No.: 17/399,643

(22) Filed: Aug. 11, 2021

(30) Foreign Application Priority Data
Aug. 26, 2020 (KR) 10-2020-0108070
Jun. 1, 2021 (KR) 10-2021-0070787

(51) Int. Cl. G06K 9/62; G06T 3/40; G06T 3/60; G16H 30/40

(52) U.S. Cl. CPC G06K 9/6256; G06K 9/6253; G06T 3/4007; G06F 3/04847; G16H 30/40; G06K 2209/09; G06T 3/60

(57) ABSTRACT

Disclosed is an apparatus for generating X-ray data including a processor that receives first image data indicating that a hidden item is hidden in a non-hidden item, and second image data indicating the non-hidden item, and generates output data and a buffer. The processor includes an extraction unit that extracts first hidden data corresponding to the hidden item from the first image data, a shape change unit that generates second hidden data by performing a shape change on the first hidden data, an interpolation unit that generates third image data by performing interpolation based on the second image data and the second hidden data, and a projection unit that generates the output data by projecting the third image data onto a 2D plane. The buffer stores a plurality of parameters associated with generation of the first hidden data, the second hidden data, the third image data, and the output data.

### CROSS-REFERENCE TO RELATED APPLICATIONS

This application claims priority under 35 U.S.C. § 119 to Korean Patent Application No. 10-2020-0108070 filed on Aug. 26, 2020 and No. 10-2021-0070787 filed on Jun. 1, 2021, in the Korean Intellectual Property Office, the disclosures of which are incorporated by reference herein in their entireties.

### BACKGROUND

Embodiments of the present disclosure described herein relate to generation of X-ray data, and more particularly, relate to an apparatus and method for generating X-ray data used for neural network learning for detecting a hidden item.

With the development of an artificial intelligence (AI) technology, the AI technology is being used in various fields. Because the prediction performance of a neural network used in AI is determined depending on a learning method of the neural network, it is necessary to learn the neural network by using high-quality learning data. However, when the neural network is learned by using only the data obtained in a real environment, it is difficult to obtain sufficient learning data and to secure the learning data, to which various environments are reflected.

Furthermore, as compared to general visible light images, the number of samples of X-ray images is small, and it is limited to obtain the X-ray images in various environments due to features of an imaging device. Accordingly, it is difficult to secure learning data for introducing AI in a medical field or customs identification field where X-ray images are mainly used.

### SUMMARY

Embodiments of the present disclosure provide an apparatus and method for generating X-ray data used to learn a neural network for detecting a hidden item.

According to an embodiment, an apparatus for generating X-ray data includes a processor that receives first image data indicating that a hidden item is hidden in a non-hidden item, and second image data indicating the non-hidden item, and generates output data and a buffer. The processor includes an extraction unit that extracts first hidden data corresponding to the hidden item from the first image data, a shape change unit that generates second hidden data by performing a shape change on the first hidden data, an interpolation unit that generates third image data by performing interpolation based on the second image data and the second hidden data, and a projection unit that generates the output data by projecting the third image data onto a two-dimensional (2D) plane. The buffer stores a plurality of parameters associated with generation of the first hidden data, the second hidden data, the third image data, and the output data.

According to an embodiment, a non-transitory computer-readable medium includes a program code that, when executed by a processor, causes the processor to extract first hidden data corresponding to a hidden item from first image data indicating that the hidden item is hidden in a non-hidden item, to generate second hidden data by performing a shape change on the first hidden data, to generate third image data by performing interpolation based on the second hidden data and second image data indicating the non-hidden item, and to generate output data by projecting the third image data onto a 2D plane.

According to an embodiment, a method for generating X-ray data includes extracting first hidden data corresponding to a hidden item from first image data indicating that the hidden item is hidden in a non-hidden item, generating second hidden data by performing a shape change on the first hidden data, generating third image data by performing interpolation based on the second hidden data and second image data indicating the non-hidden item, generating output data by projecting the third image data onto a 2D plane, and labeling a plurality of parameters associated with generation of the first hidden data, the second hidden data, the third image data, and the output data with respect to the output data.

### BRIEF DESCRIPTION OF THE FIGURES

The above and other objects and features of the present disclosure will become apparent by describing in detail embodiments thereof with reference to the accompanying drawings.

FIG. 1 is a block diagram illustrating a configuration of an apparatus for generating X-ray data, according to an embodiment of the present disclosure.

FIG. 2 conceptually illustrates a process of generating X-ray data, according to an embodiment of the present disclosure.

FIG. 3 illustrates an example of an interface for changing a hidden shape, according to an embodiment of the present disclosure.

FIG. 4 is a flowchart illustrating a method for generating X-ray data according to an embodiment of the present disclosure.

### DETAILED DESCRIPTION

Hereinafter, embodiments of the present disclosure will be described in detail and clearly to such an extent that an ordinary one in the art easily implements the present disclosure.

Components that are described in the detailed description with reference to the terms “unit”, “module”, “block”, “˜er or ˜or”, etc. and function blocks illustrated in drawings will be implemented with software, hardware, or a combination thereof. For example, the software may be a machine code, firmware, an embedded code, and application software. For example, the hardware may include an electrical circuit, an electronic circuit, a processor, a computer, an integrated circuit, integrated circuit cores, a pressure sensor, an inertial sensor, a microelectromechanical system (MEMS), a passive element, or a combination thereof

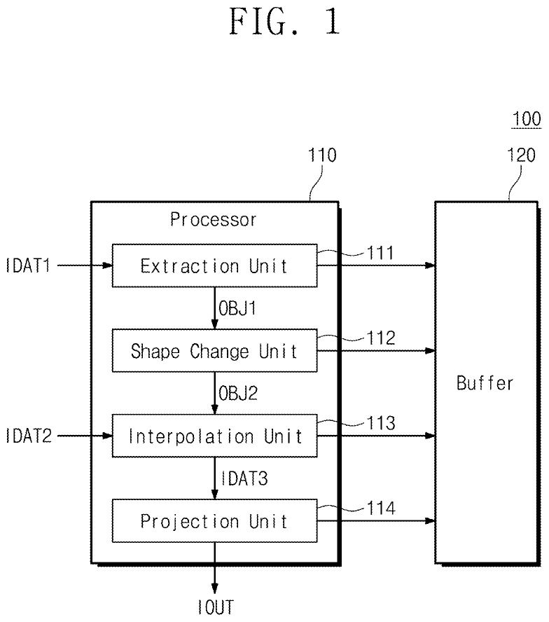

FIG. 1 is a block diagram illustrating a configuration of an apparatus 100 for generating X-ray data, according to an embodiment of the present disclosure. The apparatus 100 may include a processor 110 and a buffer 120.

The processor 110 may include an extraction unit 111, a shape change unit 112, an interpolation unit 113, and a projection unit 114. The processor 110 may receive first image data IDAT1 and second image data IDAT2. For example, the first image data IDAT1 may be 3-dimensional (3D) computed tomography (CT) data obtained by capturing a non-hidden item in a state where a hidden item (e.g., gold bars or drugs) is hidden in the non-hidden item (e.g. shoes, books, picture frames) by using a CT scheme. Furthermore, the second image data IDAT2 may be 3D CT data obtained by capturing only the non-hidden item without the hidden item.

The processor 110 may generate output data IOUT capable of being used for neural network learning for detecting a hidden item, based on the received first image data IDAT1 and the received second image data IDAT2. For example, the processor 110 may include at least one of a processing unit such as a central processing unit (CPU), a graphics processing unit (GPU), a neural network processing unit (NPU), or an accelerated processing unit (APU).

The extraction unit 111 may extract first hidden data OBJ1 corresponding to the hidden item (e.g., gold bars or drugs) from the first image data IDAT1. Specifically, the extraction unit 111 may extract the first hidden data OBJ1 by applying image segmentation to the first image data IDAT1. For example, the image segmentation may include pixel-based segmentation, edge-based segmentation, or region-based segmentation. The extraction unit 111 may use a neural network that is separately learned for the image segmentation.

The extraction unit 111 may store a plurality of parameters (e.g., a location, a size, a hidden range, or the like of an item corresponding to the first hidden data OBJ1 thus extracted), which is associated with the extraction of the first hidden data OBJ1 from the first image data IDAT1, in the buffer 120.

The shape change unit 112 may generate second hidden data OBJ2 by changing the shape of the item corresponding to the first hidden data OBJ1 received from the extraction unit 111. In other words, the shape change unit 112 may perform data augmentation on the first hidden data OBJ1.

For example, the changing of a shape may include changing the size, changing the hidden range, or rotation of the item corresponding to the first hidden data OBJ1. However, the present disclosure is not limited thereto. For example, the changing of a shape may include changing other parameters associated with a shape of the item corresponding to the first hidden data OBJ1. For example, the size change may be limited so as not to exceed the size of the non-hidden item corresponding to the first hidden data OBJ1.

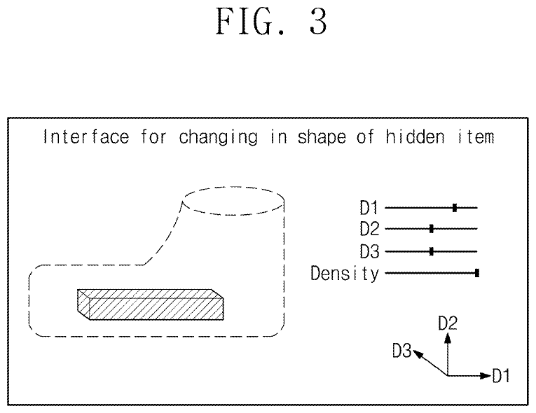

For example, the shape change unit 112 may be implemented with software stored in a non-transitory computer-readable medium, and may receive information about the size or location of an arbitrary hidden item from a user. In this case, the shape change unit 112 may provide a user interface for receiving parameters associated with a shape change. An example of a user interface provided by the shape change unit 112 is described with reference to FIG. 3.

The shape change unit 112 may store a plurality of parameters (e.g., sizes of an item corresponding to data before and after the change in size, an angle at which the item corresponding to the data is rotated, or the like) associated with the shape change of the item corresponding to the first hidden data OBJ1 in the buffer 120. Furthermore, the shape change unit 112 may learn a plurality of parameters mainly set by the user, and may variously change the shape of the item corresponding to the first hidden data OBJ1 by itself without a user input.

The interpolation unit 113 may perform interpolation based on the second image data IDAT2 on a space inside the non-hidden item, which is changed as the shape of the item corresponding to the first hidden data OBJ1 is changed to a shape of an item corresponding to the second hidden data OBJ2 in the first image data IDAT1. The interpolation unit 113 may output the interpolation result as the third image data IDAT3. That is, the third image data IDAT3 may correspond to an image in which the shape-changed hidden item corresponding to the second hidden data OBJ2 is hidden in the non-hidden item.

Through the interpolation, the shape of an image corresponding to 3D CT data obtained by capturing the non-hidden item in which a hidden item is actually hidden may be similar to a shape, in which the hidden item is hidden, and a shape of an empty space other than the hidden item, in the third image data IDAT3. The interpolation unit 113 may use a neural network that is separately learned for the image interpolation.

The interpolation unit 113 may store a plurality of parameters (e.g., a location of an internal space changed due to a shape change of the hidden item, or the like), which are associated with interpolation for a space inside the non-hidden item, in the buffer 120.

The projection unit 114 may project the third image data IDAT3 onto a two-dimensional plane. For example, the projection unit 114 may set a virtual X-ray source at any location to project the third image data IDAT3 onto a two-dimensional plane. The projection unit 114 may generate, as the output data IOUT, an X-ray image obtained by irradiating a virtual X-ray toward the third image data IDAT3 from the set virtual X-ray source.

However, the present disclosure is not limited thereto. For example, the projection unit 114 may project the third image data IDAT3 onto a two-dimensional (2D) plane through various other methods. The projection unit 114 may store, in the buffer 120, a plurality of parameters (e.g., location information of a virtual X-ray source, or the like) associated with projecting the third image data IDAT3 into a 2D plane.

The buffer 120 may include a plurality of parameters associated with the extraction of a hidden item, the shape change of the hidden item, interpolation, and 2D projection, which are performed by the processor 110. The buffer 120 may label a plurality of parameters with respect to the output data IOUT as additional information. The labeled output data IOUT may be used to learn a neural network for detecting a hidden item.

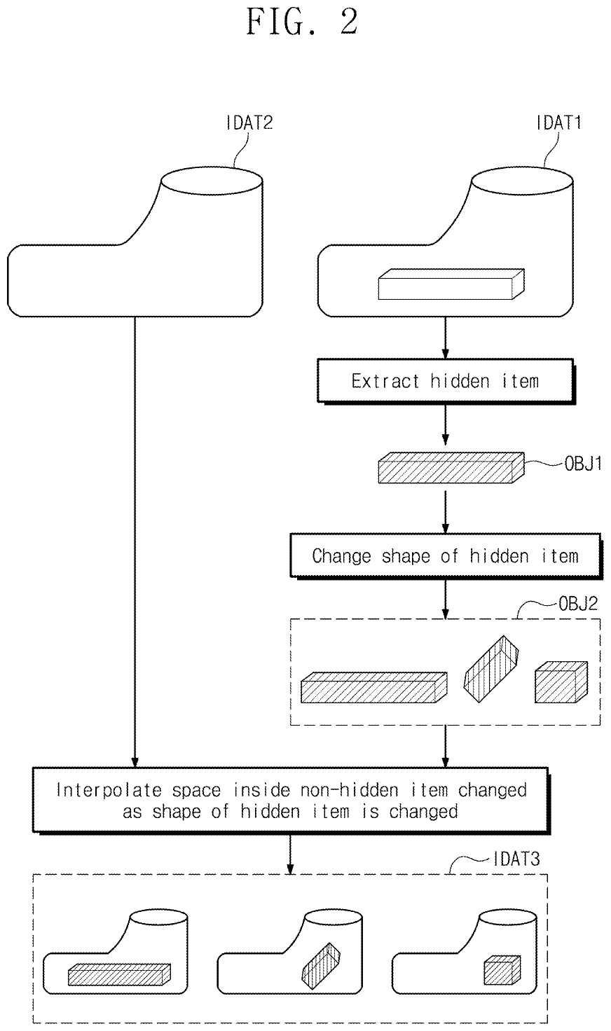

FIG. 2 conceptually illustrates a process of generating X-ray data, according to an embodiment of the present disclosure. In FIG. 2, it is assumed that a non-hidden item is a shoe and a hidden item is a gold bar. The first image data IDAT1 may indicate an image indicating that a gold bar is hidden in a shoe. The second image data IDAT2 may indicate a shoe in which no item is hidden.

As described with reference to FIG. 1, the first hidden data OBJ1 corresponding to the gold bar may be extracted from the first image data IDAT1 by the extraction unit 111. Afterward, the location, size, and hidden range of an item corresponding to the first hidden data OBJ1 may be changed by the shape change unit 112. The second hidden data OBJ2 corresponding to a gold bar, of which the shape is changed, may be generated.

The space inside the shoe, which is changed depending on a change in the shape of the gold bar inside the shoe, may be interpolated by the interpolation unit 113 based on the second image data IDAT2. The interpolated image may be output as the third image data IDAT3. In other words, the third image data IDAT3 may indicate a shoe in which the shape-changed gold bar is hidden.

FIG. 3 illustrates an example of an interface for changing a hidden shape, according to an embodiment of the present disclosure. An interface for changing a hidden shape in FIG. 3 may be an example in which the shape change unit 112 of FIG. 1 is implemented with software. As shown in FIG. 3, a user may change the size of a hidden item in each direction (D1, D2, D3) and the density of the hidden item through a slide bar.

However, the interface for changing the hidden shape in FIG. 3 is not limited to that shown in FIG. 3. It may be implemented such that the user is capable of directly entering a range of a location where the hidden item is capable of being present in each axis of D1, D2, and D3. In addition, the interface for changing the hidden shape may be implemented to change parameters (e.g., an angle at which a hidden item is rotated around a specific axis) other than the size and density of a hidden item.

As described with reference to FIG. 1, the interface for changing the hidden shape may learn pieces of information about the shape of the hidden item, which is entered by the user. Images in each of which a hidden shape is variously changed, may be automatically generated based on the learning result.

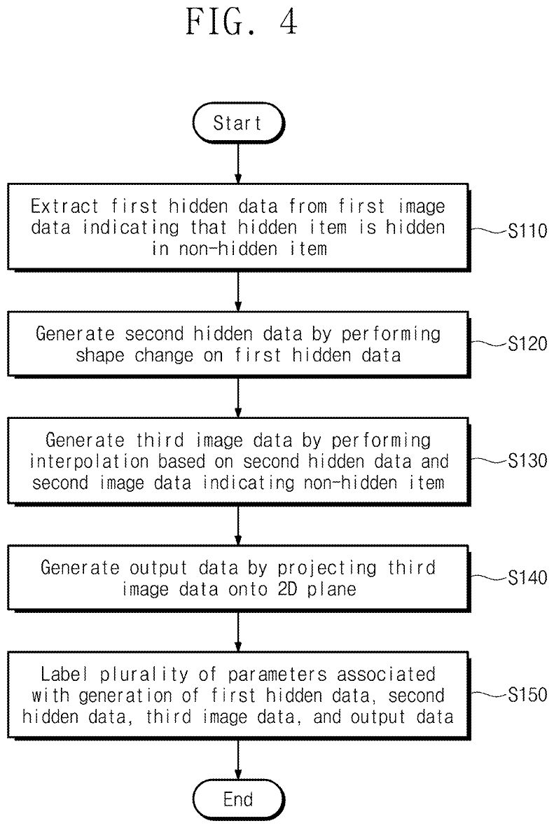

FIG. 4 is a flowchart illustrating a method for generating X-ray data according to an embodiment of the present disclosure. Hereinafter, it will be described with reference to FIG. 1 together with FIG. 4.

In operation S110, the extraction unit 111 may extract the first hidden data OBJ1 corresponding to a hidden item from the first image data IDAT1 indicating that the hidden item is hidden in a non-hidden item. For example, the first image data IDAT1 may be an image indicating that a gold bar is hidden in a shoe. The first hidden data OBJ1 may correspond to the gold bar.

In operation 5120, the shape change unit 112 may generate the second hidden data OBJ2 by performing a shape change on the first hidden data OBJ1. For example, the shape change may include a size change, a hidden range change, or a rotation of an item corresponding to the first hidden data OBJ1. However, the present disclosure is not limited thereto.

In operation S130, the interpolation unit 113 may receive the second image data IDAT2 indicating the non-hidden item, may perform interpolation on a space inside the non-hidden item that is changed depending on a change in the shape of the hidden item, and may generate the third image data IDAT3.

In operation S140, the projection unit 114 may generate the output data IOUT by projecting the third image data IDAT3 into a 2D plane. For example, the projection unit 114 may generate an X-ray image, which is obtained by irradiating a virtual X-ray toward the third image data IDAT3 from an arbitrary virtual X-ray source thus set, as the output data IOUT.

In operation S150, the buffer 120 may label a plurality of parameters associated with generation of the first hidden data OBJ1, the second hidden data OBJ2, the third image data IDAT3, and the output data IOUT with respect to the output data IOUT. The labeled output data IOUT may be provided as input data for the learning of a neural network for detecting a hidden item.

The method for generating X-rays according to an embodiment of the present disclosure may be implemented as a program code stored in a non-transitory computer-readable medium. For example, the non-transitory computer-readable media may be included in magnetic media, optical media, or combinations thereof (e.g., CD-ROM, hard drive, read-only memory, flash drive, or the like).

For example, a non-transitory computer-readable medium includes a program code that, when executed by a processor, causes the processor to extract the first hidden data OBJ1 corresponding to a hidden item from the first image data IDAT1 indicating that the hidden item is hidden in a non-hidden item, to generate second hidden data OBJ2 by performing a shape change on the first hidden data OBJ1, to generate third image data IDAT3 by performing interpolation based on the second hidden data OBJ2 and the second image data IDAT2 indicating that the non-hidden item, and to generate the output data IOUT by projecting the third image data IDAT3 onto a 2D plane.

The above description refers to embodiments for implementing the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

According to an embodiment of the present disclosure, it is possible to generate X-ray data having multiple viewpoints by projecting an image, in which a hidden item is hidden in a non-hidden item, onto a two-dimensional plane in various directions.

Furthermore, according to an embodiment of the present disclosure, a user may easily generate X-ray data for learning by providing an interface for changing a shape of the hidden item.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. An apparatus for generating X-ray data, the apparatus comprising:
a processor configured to receive first image data indicating that a hidden item is hidden in a non-hidden item, and second image data indicating the non-hidden item, and to generate output data; and
a buffer,
wherein the processor includes:
an extraction unit configured to extract first hidden data corresponding to the hidden item from the first image data;
a shape change unit configured to generate second hidden data by performing a shape change on the first hidden data;
an interpolation unit configured to generate third image data by performing interpolation based on the second image data and the second hidden data; and
a projection unit configured to generate the output data by projecting the third image data onto a two-dimensional (2D) plane, and
wherein the buffer stores a plurality of parameters associated with generation of the first hidden data, the second hidden data, the third image data, and the output data.

2. The apparatus of claim 1, wherein the shape change includes a change in movement of the hidden item corresponding to the first hidden data, a size of the hidden item corresponding to the first hidden data, and a density of the hidden item corresponding to the first hidden data.

3. The apparatus of claim 1, wherein the interpolation is performed on a space inside the non-hidden item, which is changed as the first hidden data is changed to the second hidden data.

4. The apparatus of claim 1, wherein the projection unit sets one or more virtual X-ray sources outside the third image data, and
wherein the output data is one or more images generated by irradiating X-ray from the one or more virtual X-ray sources.

5. The apparatus of claim 1, wherein the buffer labels the plurality of parameters with respect to the output data.

6. The apparatus of claim 1, wherein the shape change unit learns the plurality of parameters associated with the shape change and generates the second hidden data based on a result of the learning.

7. A non-transitory computer-readable medium including a program code that, when executed by a processor, causes the processor to:
extract first hidden data corresponding to a hidden item from first image data indicating that the hidden item is hidden in a non-hidden item;
generate second hidden data by performing a shape change on the first hidden data;
generate third image data by performing interpolation based on the second hidden data and second image data indicating the non-hidden item; and
generate output data by projecting the third image data onto a 2D plane.

8. The non-transitory computer-readable medium of claim 7, wherein the shape change includes a change in movement of the hidden item corresponding to the first hidden data, a size of the hidden item corresponding to the first hidden data, and a density of the hidden item corresponding to the first hidden data.

9. The non-transitory computer-readable medium of claim 8, wherein the processor provides an interface for receiving a plurality of parameters associated with the shape change, and
wherein the interface is used to learn the plurality of parameters and to generate the second hidden data based on a result of the learning.

10. The non-transitory computer-readable medium of claim 7, wherein the interpolation is performed on a space inside the non-hidden item, which is changed as the first hidden data is changed to the second hidden data.

11. A method for generating X-ray data, the method comprising:
extracting first hidden data corresponding to a hidden item from first image data indicating that the hidden item is hidden in a non-hidden item;
generating second hidden data by performing a shape change on the first hidden data;
generating third image data by performing interpolation based on the second hidden data and second image data indicating the non-hidden item;
generating output data by projecting the third image data onto a 2D plane; and
labeling a plurality of parameters associated with generation of the first hidden data, the second hidden data, the third image data, and the output data with respect to the output data.
